(19) United States
(12) Patent Application Publication
(10) Pub. No.: US 2024/0096464 A1
(43) Pub. Date: Mar. 21, 2024

(54) My Health Caddy

(71) Applicant: Julius Musenze, Palm Springs, CA (US)

(72) Inventor: Julius Musenze, Palm Springs, CA (US)

(21) Appl. No.: 17/949,201

(22) Filed: Sep. 20, 2022

(51) Int. Cl. G16H 10/65

(52) U.S. Cl. CPC G16H 10/65; G06K 19/06112

(57) ABSTRACT

A HIPAA-compliant medical app available to patients and healthcare providers simultaneously. Participants (patients) input their health data at their leisure, including name, date of birth, insurance information, social history (smoking history, alcohol use, drug use, sexual orientation), medical history, past and current medications, prior hospitalizations, family history, and vaccination history. During subsequent visits to theft primary care clinic, emergency room or hospital visits, the healthcare providers can expeditiously and effortlessly get a comprehensive medical history and health status of a patient in need by scanning patient's phone and entering a unique code provided either by the patient or patient's family member, or by patient accepting a request on their phone to share said information. This app will not only streamline patient care and save time at medical appointments but will also help patients get their medical information in one place to better understand their entire health picture, easily update their social, medical, surgical, family, vaccination, and medication history, as well as update their medications, allergies, insurance information and other such pertinent related information.

My Health Caddy will be a HIPAA-compliant online portal with an associated app where participants can log in and securely upload their health information. Information loaded will include:
Participant Name
Participant Date of Birth
Insurance Information
Social History (Smoking History, Alcohol Use, Sexual orientation, etc)
Medical History (Past medical history)
Allergies (Allergies to foods, medications, and irritants)
Surgical History (Any previous surgical procedures)
Hospitalizations (Any prior hospitalizations)
Family History
Medications (Medications that participant is taking)
Vaccination History

This information will be HIPAA protected and double-authenticated. Participants will be able to access their information either by entering a password set by them or by using fingerprint technology to access their health information.

On the other hand, Health care providers (Hospitals, Clinics, First Responders) will be able to quickly and effortlessly get a comprehensive medical history and health status of a patient in need in minutes by scanning patient's phone and entering a unique code provided either by the patient or patient's family member, or by patient accepting a request on their phone to share said information. Emergency Medicine physicians will be able to easily obtain comprehensive health information on patients in their ER. Hospitalists will be able to get comprehensive health information from patients in minutes prior to admitting them in the hospital.

This app will not only streamline patient care and save time at medical appointments but will also help patients get their medical information in one place to better understand their entire health picture, easily update their social, medical, surgical, family, Vaccination, and medication history, as well as update their medical allergies, insurance information and other pertinent related information.

1. I claim a HIPPA-compliant medical app, comprising of:
a means for patients to easily enter or make changes to their medical information, then having said information easily transferable to healthcare providers' device and electronic medical record, either by sharing a unique code, scanning a QR code, or by app-to-app transfer between patient and provider;
a means for patients to download MyHealth Caddy app to their devices and enter their medical information such as name, date of birth, insurance information, smoking history, alcohol use, drug use, sexual orientation, medical history, past and current medications, prior hospitalizations, family history, vaccination history;
a means by which during patients' visits to medical providers such as primary care encounters, emergency room visits, urgent care visits, holistic care, hospital visits, or rehabilitation centers, the healthcare providers can expeditiously get a comprehensive medical history and health status of a patient in need by entering a unique code provided either by the patient or patient's family member, by patient accepting a request on their phone to share said information, or by use of QR code to transfer information between patient and provider's electronic medical record;
a utility for this app is to easily share medical information to authorized parties, streamline patient care, save time at medical appointments, make medical information more accurate, enable patients to get their medical information in one place, empower patients to better understand their entire health history, enable patients to easily update the above-mentioned medical information on an on-going basis, and empower caregivers to appropriately share applicable health information to appropriate healthcare providers.
